(19) United States
(12) Patent Application Publication
(10) Pub. No.: US 2011/0257358 A1
(43) Pub. Date: Oct. 20, 2011

(54) METHOD FOR PREPARING E-CAPROLACTAM FROM N-ACYL-6-AMINOCAPROIC ACID

(76) Inventors: Daniel Mink, Eupen (BE); Rudolf Philippus Maria Guit, Maastricht (NL); Monika Muller, Aachen (DE); Petronella Catharina Raemakers-Franken, Budel (NL)

(21) Appl. No.: 13/123,571

(22) PCT Filed: Oct. 9, 2009
(86) PCT No.: PCT/NL2009/050610

§ 371 Date: Jun. 28, 2011

(30) Foreign Application Priority Data
Oct. 9, 2008 (EP) 08166286.8

(51) Int. Cl. C08G 69/16; C12P 17/10; C12N 1/00; C07D 201/08

(52) U.S. Cl. 528/323; 540/538; 435/121; 435/243

(57) ABSTRACT

The invention relates to a method for preparing ε-caprolactam comprising deacylating N-acyl-6-aminocaproic acid and forming ε-caprolactam. The deacylation may be carried out chemically or biocatalytically. The invention further relates to a host cell, comprising a recombinant vector comprising a nucleic acid sequence encoding an enzyme capable of catalysing the formation of 6-aminocaproic acid from N-acyl-6-aminocaproic acid.

The invention relates to a method for preparing ε-caprolactam (hereinafter referred to as ‘caprolactam’). The invention further relates to a host cell which may be used in a method of the invention. The invention further relates to a method for preparing a polyamide.

Caprolactam is a lactam which may be used for the production of polyamide, for instance nylon-6 or nylon-6,12. Various manners of preparing caprolactam from bulk chemicals are known in the art and include the preparation of caprolactam from cyclohexanone, toluene, phenol, cyclohexanol, benzene or cyclohexane. These intermediate compounds are generally obtained from mineral oil. In view of a growing desire to prepare materials using more sustainable technology it would be desirable to provide a method wherein caprolactam is prepared from an intermediate compound that can be obtained from a biologically renewable source or at least from an intermediate compound that is converted into caprolactam using a biochemical method. Further, it would be desirable to provide a method that requires less energy than conventional chemical processes making use of bulk chemicals from petrochemical origin.

It is known to prepare caprolactam from 6-aminocaproic acid (hereinafter also referred to as ‘6-ACA’), e.g. as described in U.S. Pat. No. 6,194,572. As disclosed in WO 2005/068643, 6-ACA may be prepared biochemically by converting 6-aminohex-2-enoic acid (6-AHEA) in the presence of an enzyme having alpha,beta-enoate reductase activity. The 6-AHEA may be prepared from lysine, e.g. biochemically or by pure chemical synthesis. Although the preparation of 6-ACA via biocatalytic reduction of 6-AHEA is feasible by the methods disclosed in WO 2005/068643, the inventors have found that—under the reduction reaction conditions—6-AHEA may spontaneously and substantially irreversibly cyclise to form an undesired side-product, notably beta-homoproline. This cyclisation may be a bottleneck in the production of 6-ACA, and may lead to a considerable loss in yield.

It is an object of the invention to provide a novel method for preparing caprolactam from 6-ACA, which may, inter alia, be used for the preparation of polyamide, that can serve as an alternative for known methods.

It is a further object to provide a novel method that would overcome one or more of the drawbacks mentioned above.

One or more further objects which may be solved in accordance with the invention, will follow from the description, below.

It has now been found possible to prepare caprolactam or 6-ACA from a specific compound, which compound is biologically available.

Accordingly, the present invention relates to a method for preparing ε-caprolactam comprising deacylating N-acyl-6-aminocaproic acid and forming ε-caprolactam.

An N-acyl-6-aminocaproic acid (which may be referred to hereinafter as ‘N-Ac-ACA’) may in particular be selected from naturally available N-Ac-ACA's. In particular it may be N-acetyl-6-aminocaproic acid (N-acetyl-ACA), which compound is naturally available, e.g. from a cycad, in particular from Dioon edule, e.g. from the seeds thereof.

The invention further relates to a method for preparing 6-ACA from N-Ac-ACA, comprising deacylating N-Ac-ACA, in particular comprising deacetylating N-acetyl-ACA, in the presence of a biocatalyst catalysing such deacylation. The 6-ACA may thereafter be subjected to a cyclisation reaction to form caprolactam.

The invention further relates to a method for preparing a polyamide, comprising polymerising caprolactam obtained in a method according to the invention.

The invention further relates to a host cell, comprising a recombinant vector comprising a nucleic acid sequence encoding an enzyme having catalytic activity in the formation of 6-ACA from N-Ac-ACA, in particular from N-acetyl-ACA.

When referring herein to a carboxylic acid or a carboxylate, e.g. N-acetyl-ACA, these terms are meant to include the neutral (protonated) carboxylic acid group, the corresponding carboxylate (its conjugated base) as well as salts thereof. When referring herein to an amino acid, e.g. 6-ACA, this term is meant to include the amino acid in its zwitterionic form (in which the amino group is in the protonated and the carboxylate group in the deprotonated form) or the amino acid in which the amino group is protonated and the carboxylic group is in its neutral form or the amino acid in which the amino group is in its neutral form and the carboxylate group is in the deprotonated form as well as salts thereof.

In accordance with the invention, no problems have been noticed with respect to an undesired cyclisation of an intermediate product, e.g. an undesired cyclisation to form cyclic oligomers, when forming 6-ACA and optionally caprolactam, resulting in a loss of yield.

It is envisaged that a method of the invention allows a comparable or even better yield than the method described in WO 2005/68643. It is envisaged that a method of the invention may in particular be favourable if a use is made of a living organism.

It is further envisaged that in an embodiment of the invention the productivity of 6-ACA (g/l·h formed) in a method of the invention may be improved.

The term “or” as used herein means “and/or” unless specified otherwise.

The term “a” or “an” as used herein means “at least one” unless specified otherwise.

When referring to a noun (e.g. a compound, an additive, etc.) in singular, the plural is meant to be included.

When referring to a compound of which stereoisomers exist, the compound may be any of such stereoisomers or a combination thereof. Thus, when referred to, e.g., an amino acid of which enantiomers exist, the amino acid may be the L-enantiomer, the D-enantiomer or a combination thereof. In case a natural stereoisomer exists, the compound is preferably a natural stereoisomer.

When an enzyme is mentioned with reference to an enzyme class (EC) between brackets, the enzyme class is a class wherein the enzyme is classified or may be classified, on the basis of the Enzyme Nomenclature provided by the Nomenclature Committee of the International Union of Biochemistry and Molecular Biology (NC-IUBMB), which nomenclature may be found at http://www.chem.gmul.ac.uk/iubmb/enzyme/. Other suitable enzymes that have not (yet) been classified in a specified class but may be classified as such, are meant to be included.

If referred herein to a protein or gene by reference to a accession number, this number in particular is used to refer to a protein or gene having a sequence as found in Uniprot on 9 Oct. 2008, unless specified otherwise.

The term “homologue” is used herein in particular for polynucleotides or polypeptides having a sequence identity of at least 30%, preferably at least 40%, more preferably at least 60%, more preferably at least 65%, more preferably at least 70%, more preferably at least 75%, more preferably at least 80%, in particular at least 85%, more in particular at least 90%, at least 91%, at least 92%, at least 93%, at least 94%, at least 95%, at least 96%, at least 97%, at least 98% or at least 99%. The term homologue is also meant to include nucleic acid sequences which differ from another nucleotide sequence due to the degeneracy of the genetic code and encode the same polypeptide sequence.

Sequence identity or similarity is herein defined as a relationship between two or more polypeptide sequences or two or more nucleic acid sequences, as determined by comparing the sequences. Usually, sequence identities or similarities are compared over the whole length of the sequences, but may however also be compared only for a part of the sequences aligning with each other. In the art, “identity” or “similarity” also means the degree of sequence relatedness between polypeptide sequences or nucleic acid sequences, as the case may be, as determined by the match between such sequences. Preferred methods to determine identity or similarity are designed to give the largest match between the sequences tested. In context of this invention a preferred computer program method to determine identity and similarity between two sequences includes BLASTP and BLASTN (Altschul, S. F. et al., J. Mol. Biol. 1990, 215, 403-410, publicly available from NCBI and other sources (BLAST Manual, Altschul, S., et al., NCBI NLM NIH Bethesda, Md. 20894). Preferred parameters for polypeptide sequence comparison using BLASTP are gap open 10.0, gap extend 0.5, Blosum 62 matrix. Preferred parameters for nucleic acid sequence comparison using BLASTN are gap open 10.0, gap extend 0.5, DNA full matrix (DNA identity matrix).

In a method of the invention, a biocatalyst may be used. Thus, in such a method at least one reaction step in the method is catalysed by a biological material or moiety derived from a biological source, for instance an organism or a biomolecule derived there from. The biocatalyst may in particular comprise one or more enzymes.

The biocatalyst may be used in any form. In an embodiment, one or more enzymes are used isolated from the natural environment (isolated from the organism it has been produced in), for instance as a solution, an emulsion, a dispersion, a suspension (e.g. of suspended freeze-dried biocatalyst), a lysate, a cell-free extract, or immobilised on a support.

In an embodiment, one or more enzymes form part of a living organism (such as living whole cells). The enzymes may perform a catalytic function inside the cell. It is also possible that the enzyme may be secreted into a medium, wherein the cells are present.

Living cells may be growing cells, resting or dormant cells (e.g. spores) or cells in a stationary phase. It is also possible to use an enzyme forming part of a permeabilised cell (i.e. made permeable to a substrate for the enzyme or a precursor for a substrate for the enzyme or enzymes).

A biocatalyst used in a method of the invention may in principle be any organism, or be obtained or derived from any organism. The organism may be eukaryotic or prokaryotic. In particular the organism may be selected from animals (including humans), plants, bacteria, archaea, yeasts and fungi. A suitable biocatalyst for catalysing the deacylation may in particular be selected from the group of biocatalysts comprising an enzyme catalysing said deacylation originating from an organism selected from the group of Candida, Mycobacterium, Alcaligenes, more in particular from the group of Candida cylindracea, Candida rugosa, Mycobacterium neoaurum and Alcaligenes faecalis.

The biocatalyst may be a heterologous biocatalyst, in particular a heterologous cell. A heterologous biocatalyst, is a biocatalyst comprising a heterologous protein or a heterologous nucleic acid (usually as part of the cell's DNA or RNA) The term “heterologous” when used with respect to a nucleic acid sequence (DNA or RNA), or a protein refers to a nucleic acid or protein that does not occur naturally as part of the organism, cell, genome or DNA or RNA sequence in which it is present, or that is found in a cell or location or locations in the genome or DNA or RNA sequence that differ from that in which it is found in nature. It is understood that heterologous DNA in a heterologous organism is part of the genome of that heterologous organism. Heterologous nucleic acids or proteins are not endogenous to the cell into which they are introduced, but have been obtained from another cell or synthetically or recombinantly produced. Generally, though not necessarily, such nucleic acids encode proteins that are not normally produced by the cell in which the DNA is transcribed or expressed. Similarly heterologous RNA encodes for proteins not normally expressed in the cell in which the heterologous RNA is present. Heterologous nucleic acids and proteins may also be referred to as foreign nucleic acids or proteins. Any nucleic acid or protein that one of skill in the art would recognise as heterologous or foreign to the cell in which it is expressed is herein encompassed by the term heterologous nucleic acid or protein.

When referred to an enzyme or another biocatalytic moiety from a particular source, recombinant enzymes or other recombinant biocatalytic moieties, originating from a first organism, but actually produced in a (genetically modified) second organism, are specifically meant to be included as enzymes or other biocatalytic moieties, from that first organism.

In accordance with the invention it is possible to convert N-Ac-ACA to caprolactam without needing a biocatalyst or another catalyst. Caprolactam can be prepared chemically by hydrolysis (to remove the acetic acid group) and cyclisation, under pressure at elevated temperature. Both hydrolysis and cyclisation can be carried out in the same reactor, if desired.

In particular, a (thermal) chemical conversion of N-Ac-ACA to caprolactam may be carried out in a so-called high pressure cyclisation process. In this process an aqueous mixture of N-Ac-ACA is heated at a temperature in the range of 200 to 350° C., preferably in the range 270 to 330° C., and most preferably the temperature is higher than 280° C. or more.

The pressure is preferably between 5 and 20 MPa. Normally this pressure will be higher than or equal to the resulting pressure of the liquid reaction mixture at the temperature employed. Further reaction conditions, such as suitable N-Ac-ACA feed concentrations and reaction residence times can routinely be optimized by the person skilled in the art, based on common general knowledge and the present disclosure.

In a further specific embodiment, caprolactam is prepared from N-Ac ACA making use of a so-called solvent free cyclisation process. Herein a melt of N-Ac-ACA, usually further comprising sufficient water to allow the hydrolysis to proceed, but otherwise generally not containing any solvents, is allowed to react at a temperature in the range of from 250 to 400° C. and preferably in the range of from 270 to 350° C. Compared to the high pressure cyclisation process the pressure is much lower and is typically in the range of from 0.5 to 2 MPa. Caprolactam that has formed is usually steam-stripped from the reaction mixture. Steam may be added as such and/or may be generated in situ from water present in the feed. After leaving the reaction zone the vapour product mixture containing caprolactam is condensed. To optimize the reaction rate and the caprolactam concentration in the condensate the amount of added steam and/or water can routinely be optimized by the person skilled in the art.

In both the solvent free and high pressure cyclisation processes unconverted intermediates may be recycled. The advantages of the solvent free cyclisation process over the high pressure cyclisation process are that a very high one pass caprolactam yield is easily obtained, that recycle of unconverted intermediates may not be necessary and that product recovery may be cheaper.

In a specific embodiment, N-Ac-ACA is first deacylated thereby forming 6-ACA, after which 6-ACA may be cyclised in a separate process step. This allows removal of acid, formed as a result of the deacylation (or, more specifically, deacetylation by removal of acetic acid in case N-acetyl aminocaproic acid is deacylated), to form 6-ACA, prior to cyclising 6-ACA, during or after the deacylation process. Such removal may be an actual separation of the formed acid (such as acetic acid) from the 6-ACA, or—in case use is made of a whole cell biotransformation or the like, the acid may act as a carbon source for the cell, and thus be removed as a result of a bioconversion. The removal of acid, in particular acetic acid, is considered advantageous as it is contemplated that acid may detrimentally interfere with the cyclisation process, or at least may have a detrimental effect on the total yield, as the hydrolysis is an equilibrium reaction. By removing the acid formed in the deacylation prior to the cyclisation, esterification of 6-ACA with the acid during the cyclisation is avoided, or at least substantially reduced. In principle, it is also possible to remove the acid continuously, in order to keep the equilibrium of the hydrolysis to the right side.

It is further contemplated that the acid, in particular acetic acid, may be more efficiently removed from 6-ACA than from caprolactam.

In an advantageous method of the invention, the deacylation is biocatalysed. In particular use may be made of a hydrolase (EC 3) having catalytic activity with respect to the deacylation of N-Ac-ACA, more in particular with respect to the deacetylation of N-acetyl-ACA. The hydrolase preferably is selected from the group of hydrolases acting on ester bonds (lipases, esterases) (EC 3.1), peptide hydrolases acting on peptide bonds (peptidase, proteinases) (EC 3.4), and hydrolases acting on C—N bonds other than peptide bonds (EC 3.5).

In particular a hydrolase acting on C—N bonds other than peptide bonds may be selected from the group of amidases acting on linear amides (EC 3.5.1), specifically from the group of amino amidases, more specifically from the group of amino amidases from Mycobacterium, more specifically amino amidases from Mycobacterium neoaurum.

In particular a hydrolase acting on C—N bonds other than peptide bonds may be selected from the group of amidases acting on linear amides (EC 3.5.1), specifically acylases, specifically penicillin acylase (also named penicillin amidase) (EC 3.5.1.11), more specifically penicillin acylase from Alcaligenes, more specifically penicillin G acylase from Alcaligenes faecalis.

In particular, good results have been achieved with a biocatalyst comprising an enzyme comprising an amino acid sequence according to Sequence ID 2, or a homologue thereof.

Use may be made of a host cell, capable of producing an enzyme for catalysing the deacylation.

A host cell according to the invention comprises a recombinant vector comprising a nucleic acid sequence encoding an enzyme having catalytic activity with respect to the formation of 6-aminocaproic acid from N-acyl-6-aminocaproic acid, in particular from N-acetyl-6-aminocaproic acid. The enzyme may in particular comprise an amino acid sequence according to Sequence ID 2, or a homologue thereof. More in particular the enzyme may be encoded by a nucleic acid sequence according to Sequence ID 1 or a homologue thereof.

Reaction conditions for any biocatalytic step in the context of the present invention may be chosen depending upon known conditions for the biocatalyst, in particular the enzyme, the information disclosed herein and optionally some routine experimentation.

In principle, the pH of the reaction medium used may be chosen within wide limits, as long as the biocatalyst is active under the pH conditions. Alkaline, neutral or acidic conditions may be used, depending on the biocatalyst and other factors, as will be understood by the skilled person. In case the method includes the use of a micro-organism, e.g. for expressing an enzyme catalysing a method of the invention, the pH is selected such that the micro-organism is capable of performing its intended function or functions. The pH may in particular be chosen within the range of four pH units below neutral pH and two pH units above neutral pH, i.e. between pH 3 and pH 9 in case of an essentially aqueous system at 25° C. A system is considered aqueous if water is the only solvent or the predominant solvent (>50 wt. %, in particular >90 wt. %, based on total liquids), wherein e.g. a minor amount of alcohol or another solvent (<50 wt. %, in particular <10 wt. %, based on total liquids) may be dissolved (e.g. as a carbon source) in such a concentration that micro-organisms which may be present remain active. In particular in case a yeast and/or a fungus is used, acidic conditions may be preferred, in particular the pH may be in the range of pH 3 to pH 8, based on an essentially aqueous system at 25° C. If desired, the pH may be adjusted using an acid and/or a base or buffered with a suitable combination of an acid and a base.

In principle, the incubation conditions can be chosen within wide limits as long as the biocatalyst shows sufficient activity and/or growth. This includes aerobic, micro-aerobic, oxygen limited and anaerobic conditions.

Anaerobic conditions are herein defined as conditions without any oxygen or in which substantially no oxygen is consumed by the biocatalyst, in particular a micro-organism, and usually corresponds to an oxygen consumption of less than 5 mmol/l·h, in particular to an oxygen consumption of less than 2.5 mmol/l·h, or less than 1 mmol/l·h.

Aerobic conditions are conditions in which a sufficient level of oxygen for unrestricted growth is dissolved in the medium, able to support a rate of oxygen consumption of at least 10 mmol/l·h, more preferably more than 20 mmol/l·h, even more preferably more than 50 mmol/l·h, and most preferably more than 100 mmol/l·h.

Oxygen-limited conditions are defined as conditions in which the oxygen consumption is limited by the oxygen transfer from the gas to the liquid. The lower limit for oxygen-limited conditions is determined by the upper limit for anaerobic conditions, i.e. usually at least 1 mmol/l·h, and in particular at least 2.5 mmol/l·h, or most specifically at least 5 mmol/l·h. The upper limit for oxygen-limited conditions is determined by the lower limit for aerobic conditions, i.e. less than 100 mmol/l·h, less than 50 mmol/l·h, less than 20 mmol/l·h, or less than to 10 mmol/l·h.

Whether conditions are aerobic, anaerobic or oxygen limited is dependent on the conditions under which the method is carried out, in particular by the amount and composition of ingoing gas flow, the actual mixing/mass transfer properties of the equipment used, the type of micro-organism used and the micro-organism density.

In principle, the temperature used is not critical, as long as the biocatalyst, in particular the enzyme, shows substantial activity. Generally, the temperature may be at least 0° C., in particular at least 15° C., more in particular at least 20° C. A desired maximum temperature depends upon the biocatalyst. In general such maximum temperature is known in the art, e.g. indicated in a product data sheet in case of a commercially available biocatalyst, or can be determined routinely based on common general knowledge and the information disclosed herein. The temperature is usually 90° C. or less, preferably 70° C. or less, in particular 50° C. or less, more in particular or 40° C. or less.

In particular if a biocatalytic reaction is performed outside a host organism, a reaction medium comprising an organic solvent may be used in a high concentration (e.g. more than 50 wt. %, or more than 90 wt. %, based on total liquids), in case an enzyme is used that retains sufficient activity in such a medium.

The invention will now be illustrated by the following examples.

### EXAMPLES

### Chemicals

N-acetyl-6-aminocaproic acid (N-acetyl-ACA) was obtained from Acros Organics (Purity 99%; Catalogue number 186570250).

### Example 1

### Formation of Caprolactam from N-Ac-ACA by High Pressure Cyclisation

### 1.1. HPLC-UV Analysis Method for Detection of N-Ac-ACA, CAP, 6-ACA, and Oligomers

For HPLC analysis a 250×3 mm Zorbax SB-C₁₈ column (5 μm, 80 Å, temperature 40° C.) was used applying sandwich injection (2 μl formic acid zone—3 μl sample—2 μl formic acid zone). Mobile phases, A: 10 mM H₃PO₄, pH 2.6 (NaOH); B: acetonitrile; gradient t_{0 min.} 99% A—1% B, t_{36 min.} 68% A—32% B. Flow, 0.51 ml/min. UV detection at λ=200 and 220 nm. At t_{31 min.} post gradient injection of 250 μl formic acid. Post-column reaction detection using the following reagent: 38 g sodium tetraborate, 3 g sodium hydroxide and 1 ml 3-mercaptopropionic acid dissolved in 1 l water to which 0.8 g ortho-phthalaldehyde in 20 ml methanol is added. Post-column flow-rate, 0.25 ml/min; polyether ether ketone (PEEK) capillary 0.25 mm×3 m. Fluorescence detection applying excitation at 330 nm (λₑₓ=330 nm), and emission at 420 nm (λₑₘ=420 nm). At t_{31 min,} injection of 250 μl formic acid to clean the stationary phase.

1.2 Formation of Caprolactam from N-Ac-ACA by High Pressure Cyclisation

A high pressure cyclisation process was used to convert N-acetyl-ACA into caprolactam “Carius” tubes.

Two 58 ml glass inserts for the Carius tubes were each filled with approximately 23 grams of an aqueous solution containing 5.1 wt % N-acetyl-ACA (analysed by HPLC). After inertisation with nitrogen the contents of the tubes were heated to the desired process conditions of 300° C. At this temperature the equilibrium pressure above the solution became approximately 91 bar. Both tubes were held for 1 hour at 300° C. After 1 hour the Carius tubes were cooled down to “freeze” the equilibrium product mixture. The starting solution and the product solutions were analysed by HPLC.

Table 1 shows the HPLC analysis results of samples from the starting solution and from both test-tubes after reaction. It can be calculated from these analysis results that the product solutions were concentrated during the experiment by approximately 19±2% (due to evaporation of water during the experiment). The HPLC chromatograms showed a clean analysis result for caprolactam, 6-ACA and “normal” N6-oligomers, without any indication for the presence of unwanted by-products.

TABLE 1
HPLC analysis results from high pres-
sure cyclisation experiments (in mol/l)

| | N-acetyl- | Capro- | 6- | Linear | Linear | Linear |
|---|---|---|---|---|---|---|
| * | ACA | lactam | ACA | dimer | trimer | tetramer |
| Starting | 0.294 | 0 | 0 | 0 | 0 | 0 |
| solution | | | | | | |
| Experiment 1 | 0.0428 | 0.264 | 0.0296 | 0.0061 | 0.00075 | 0.00014 |
| Experiment 2 | 0.0382 | 0.285 | 0.0281 | 0.0054 | 0.00065 | 0.00012 |

*Cyclic oligomers were below the detection limit. Of the linear oligomers only oligomers ≦ 4 were above the detection limit.

### Example 2

### Biocatalytic Synthesis of 6-ACA from N-Ac-ACA

### 2.1 LC-MS Analysis Method for the Detection of 6-Aminocaproic Acid Formed in Enzymatic Hydrolysis

Sample Preparation:

Samples were diluted 1:1 with eluent A.

Experiments were performed on the SCIEX AP1150 MS from Applied Biosystems.

### LC Conditions:

Column: 50×4.6 mm Nucleosil C18, 5 μm (Machery & Nagel) pre column coupled to a 50×4.6 mm id. Prevail C18, 5 μm (Alltech).
Column temperature: room temperature
Eluent: A: water containing 0.1% formic acid
   B: acetonitrile containing 0.1% formic acid
Gradient: The gradient was started at 95% (v/v) A, changed in 1.8 min. to 70% (v/v) B, changed in 0.1 min to 95% (v/v) A (t=1.9 min.).
   From 1.9 to 4 min. the gradient was held to 95% (v/v) B.
Flow: 1.8 ml/min, before entering the MS the flow is split 1:5
Injection volume: 1 μl

### MS Conditions:

Ionisation: positive ion turbo ion spray
   source conditions:
      ion spray voltage: 5 kV
      temperature: 350° C.
      defragmentation potential: 16V
      focusing potential: 140V
Scan mode: selective ion mode: m/z 132.1 (dwell time 200 msec)
   Under the applied conditions 6-ACA elutes at 0.87 minutes.

### 2.2 Preparation of Biocatalysts

The following biocatalysts were used to catalyse the conversion of N-acetyl-ACA into 6-ACA: A) freeze-dried cell free extract of recombinant penicillin acylase from Alcaligenes faecalis produced in Escherichia coli and B) Mycobacterium neoaurum freeze-dried cells.

Biocatalyst A was obtained by PCR amplification of the wild type gene (Sequence ID 1) from chromosomal DNA of Alcaligenes faecalis (ATCC19018), cloning into an expression vector and recombinant expression of the enzyme (Sequence ID 2) in Escherichia coli as reported e.g. in “Applied and Environmental Microbiology 1997, 63(9), 3412-3418” and EP0453047.

Biocatalyst B was obtained by growth of Mycobacterium neoaurum strain ATCC 25795 under following conditions. One litre of Mycomed medium containing 4.8 g/l nitrilotriacetic acid (NTA), 4 g/l urea, 6 g/l glucose, 20 g/l yeast carbon base (YCB from Difco), 1.55 g/l K₂HPO₄ and 0.85 g/l NaH₂PO₄.H₂O were adjusted to pH 7 and inoculated with a glycerol stock culture of Mycobacterium neoaurum strain ATCC 25795. The preculture was shaken on a New Brunswick Scientific G53 shaker (150 rpm, amplitude 4 cm) at 37° C. for 168 hours. When an optical density (OD_{620 nm}) of 3.45 was reached, 500 ml of the preculture was used to inoculate 9 l of Mycomed medium. Amidase expression was induced by NTA present in the Mycomed medium. The fermentation culture was stirred at 375-750 rpm at an aeration rate of 0.5-2 l/min. The pH was kept constant at 7 by addition of H₃PO₄ and NaOH. The cultivation temperature was 37° C. After 44 hours of cultivation, the culture was feeded by addition of 10 g/l YCB. After 68 hours of cultivation, the culture was feeded by addition of 10 g/l glucose. After 94 hours of cultivation, the culture was harvested by centrifugation at 12,000 g for 10 minutes. The cell pellet was washed in 20 mM HEPES/NaOH buffer, pH 7 and subsequently freeze-dried for storage.

2.3 Biocatalytic Synthesis of 6-ACA from N-Ac-ACA

Bioconversions were done in 96 well microtiterplate format. The biocatalysts were present in lyophilized form and suspended in 100 μl 50 mM potassium phosphate buffer, pH 7.5 before use yielding in a final concentration of 40 mg/well (biocatalyst A), or 2 mg/well (biocatalyst B).

The enzymatic reactions were started by addition of 150 μl of a 133 mM N-acetyl-ACA solution in 50 mM potassium phosphate buffer pH 7.5. The pH of the reaction mixtures was adjusted to pH 7.5 by addition of 15 μl of 1M KOH. The mixtures were incubated overnight at 28° C. while shaking at 500 rpm on an IKA orbital shaker. Thereafter, reactions were stopped and diluted 4 times by addition of 750 μl of a 50/50 mixture of water/acetonitrile, containing 0.5% formic acid. The reaction mixtures were centrifuged at 4000 rpm for 30 minutes and analysed by means of LC-MS.

6-ACA was detected in the presence of the biocatalyst in a concentration of 0.97 mmol/l (enzyme A), and 0.09 mmol/l (enzyme B).

The biocatalytically formed 6-ACA can be cyclised to form caprolactam in a manner known per se.

1. Method for preparing ε-caprolactam comprising deacylating N-acyl-6-aminocaproic acid and forming ε-caprolactam.

2. Method according to claim 1, comprising chemically deacylating N-acyl-6-aminocaproic acid.

3. Method according to claim 1, comprising biocatalytically deacylating N-acyl-6-aminocaproic acid, thereby forming 6-aminocaproic acid, and cyclising 6-aminocaproic acid, thereby forming ε-caprolactam.

4. Method according to claim 3, wherein the biocatalytic deacylation is carried out in the presence of biocatalyst comprising a hydrolase selected from the hydrolases acting on ester bonds (EC 3.1), peptide hydrolases acting on peptide ponds (EC 3.4), and hydrolases acting on C—N bonds other than peptide bonds (EC 3.5), in particular in the presence of a biocatalyst comprising an amidase acting on linear amides (EC 3.5.1), more in particular a penicillin acylase (EC 3.5.1.11).

5. Method according to claim 3, wherein the biocatalytic deacylation is carried out in the presence of a biocatalyst comprising an enzyme catalysing said deacylation originating from an organism selected from the group of Candida, Mycobacterium and Alcaligenes, in particular from the group of Candida cylindracea, Candida rugosa, Mycobacterium neoaurum and Alcaligenes faecalis.

6. Method according to claim 3, wherein the biocatalyst comprises an enzyme comprising an amino acid sequence according to Sequence ID 2, or a homologue thereof.

7. Method according to claim 1, wherein the forming of caprolactam is carried out by means of a solvent free cyclisation reaction at a pressure in the range of from 0.5 to 2 MPa and at a temperature in the range of from 250 to 400° C., or by means of a high pressure cyclisation reaction at a pressure between 5 and 20 MPa and at a temperature in the range of from 200 to 350° C.

8. Method according to claim 1 wherein the N-acyl-6-aminocaproic acid is N-acetyl-6-aminocaproic acid.

9. Method according to claim 1, wherein the N-acyl-6-aminocaproic acid is obtained from a biological source.

10. Method according to claim 9, wherein the biological source is a cycad, in particular Dioon edule.

11. Method for preparing a polyamide, comprising polymerising ε-caprolactam obtained in a method according to claim 1.

12. Host cell, comprising a recombinant vector comprising a nucleic acid sequence encoding an enzyme capable of catalysing the formation of 6-aminocaproic acid from N-acyl-6-aminocaproic acid.

13. Host cell according to claim 12, wherein the nucleic acid sequence encoding an enzyme capable of catalysing the formation of 6-aminocaproic acid from N-acyl-6-aminocaproic acid is a nucleic acid sequence encoding an enzyme capable of catalysing the formation of 6-aminocaproic acid from N-acetyl-6-aminocaproic acid.

14. Host cell according to claim 12, wherein the enzyme is an enzyme as defined in claim 6.

15. Host cell according to claim 14, wherein the enzyme is encoded by a nucleic acid sequence according to Sequence ID 1 or a homologue thereof.


SEQUENCE LISTING (2 sequences)

SEQ ID NO: 1 - DNA, 2448 bp, Alcaligenes faecalis
  CDS (1)..(2448)
atgcagaaagggcttgttcgtaccgggcttgtggccgctggtttgatcttgggttgggcgggggcaccgacccacgcgcaagtgcagtcggtagaggtgatgcgggacagttatggcgtgccgcacgtctttgccgacagccactatggcttgtattacggctatggttatgcggtcgcccaagaccgtctgttccagatggacatggcgcgtcgctcctttgtcggcacaaccgccgccgtcttaggccctggtgagcaagatgcctacgtcaagtacgacatgcaggtgcggcagaacttcaccccggcttccatacagcggcagatcgcggccttgtccaaggatgagcgcgatatttttcgtggctatgccgatggctataacgcctatctggagcaggtgcggcgtcgccctgagttgctgcccaaagaatatgtggattttgatttccagcccgagccgctgaccgactttgatgtggtcatgatctgggtgggctccatggccaatcgcttctccgacacgaatctggaagtgacggcactggccatgcgtcagtctctggagaaacagcacggcccggaacgaggccgtgccttgtttgatgagctgctgtggatcaatgacacaacagctcccactacggttccggcccccgctgccgagcacaagccgcaggcacaagcagggacgcaggatctggctcatgtttcctcgccagtactggctaccgagctagagcgccaggacaagcactggggcggccgtggcccggacttcgcgcccaaggctagcaacctgtggagcactcgccccgagcgagtgcaggagggctcgaccgtactgatcaacggcccacagtttggctggtacaacccggcctacacctatggcattggcttgcatggcgccggcttcgatgtggtgggtaatacgccttttgcctatccgatcgtactgtttggcaccaatagcgagattgcctggggggcgactgctggcccgcaagatgtggtggacatatatcaggaaaaattgaacccctcgcgtgccgatcagtactggttcaacaatgcctggcgcacgatggagcagcgcaaggaacgtatccaggtacgcggtcaggctgatcgggaaatgacgatctggcgcaccgtgcacggccctgtgatgcagtttgattacgatcagggcgcggcgtacagcaagaaacgcagctgggatggctatgaggtgcagtccttgctagcctggttgaacgtggccaaggcccgcaactggacggagtttctggatcaagccagcaagatggcgatttcgatcaactggtactacgccgacaagcacggcaatattggttatgtctcgccggccttcctgccccagcgtcctgccgatcaggacatccgtgtccctgccaagggggatggcagcatggagtggctgggcatcaagagtttcgacgcgattcccaaagcctacaatccaccccagggctatctggtcaactggaacaacaagcctgcgccggacaaaaccaatacggatacttactattggacctatggcgaccgcatgaatgaactggtcagtcagtaccagcagaaagacctcttcagtgtgcaggagatctgggagttcaatcaaaaagcctcctatagcgatgtgaactggcgctacttccgcccacatctggaaaagctggcgcaacagctgccggccgacgatagcagcaaggcggcgctgacgatgttgctcgcctgggatggaatggaacaggatcagggagggcaaaatgccggaccggcgcgggtgctcttcaagacctggctggaagaaatgtacaagcaggtcttgatgccggtggtgcctgaatcgcatcgcgccatgtatagccagactggttttgccacgcagcaaggtcccaaccccggttccatcaacttgagcatgggcaccaaggtcttgttgcgtgccttggtgctggaagcccatcccgatcccaagcgtgtgaatgtctttggtgagcgttcgtctcaggaaatcatgcacacagctttgcaaaatgcgcaggcccgcttgagccaggagcagggcgctcagatggcgcgctggaccatgccgacctccgtgcatcgtttcagcgacaagaacttcacgggaaccccgcagacgatgcctggcaatacctttgcctttaccggctatcagaatcgaggcacggaaaataaccgcgtggtgtttgatgccaagggcgtggagttctgcgacgccatgccgcccggccaaagcggtttcaccgaccgcaatggagtgcgcagcccgcattatgaggatcagctgaagttgtacgagaacttcgagtgcaagacgatggatgtgacgcatgcggacattcgtcgtaatgcgcaaagcagcacgatgctgttgattcagcctcagcct

SEQ ID NO: 2 - protein, 816 aa, Alcaligenes faecalis
MQKGLVRTGLVAAGLILGWAGAPTHAQVQSVEVMRDSYGVPHVFADSHYGLYYGYGYAVAQDRLFQMDMARRSFVGTTAAVLGPGEQDAYVKYDMQVRQNFTPASIQRQIAALSKDERDIFRGYADGYNAYLEQVRRRPELLPKEYVDFDFQPEPLTDFDVVMIWVGSMANRFSDTNLEVTALAMRQSLEKQHGPERGRALFDELLWINDTTAPTTVPAPAAEHKPQAQAGTQDLAHVSSPVLATELERQDKHWGGRGPDFAPKASNLWSTRPERVQEGSTVLINGPQFGWYNPAYTYGIGLHGAGFDVVGNTPFAYPIVLFGTNSEIAWGATAGPQDVVDIYQEKLNPSRADQYWFNNAWRTMEQRKERIQVRGQADREMTIWRTVHGPVMQFDYDQGAAYSKKRSWDGYEVQSLLAWLNVAKARNWTEFLDQASKMAISINWYYADKHGNIGYVSPAFLPQRPADQDIRVPAKGDGSMEWLGIKSFDAIPKAYNPPQGYLVNWNNKPAPDKTNTDTYYWTYGDRMNELVSQYQQKDLFSVQEIWEFNQKASYSDVNWRYFRPHLEKLAQQLPADDSSKAALTMLLAWDGMEQDQGGQNAGPARVLFKTWLEEMYKQVLMPVVPESHRAMYSQTGFATQQGPNPGSINLSMGTKVLLRALVLEAHPDPKRVNVFGERSSQEIMHTALQNAQARLSQEQGAQMARWTMPTSVHRFSDKNFTGTPQTMPGNTFAFTGYQNRGTENNRVVFDAKGVEFCDAMPPGQSGFTDRNGVRSPHYEDQLKLYENFECKTMDVTHADIRRNAQSSTMLLIQPQP